(19) United States
(12) Patent Application Publication
(10) Pub. No.: US 2026/0284269 A1
(43) Pub. Date: Sep. 24, 2026

(54) HYDROGEL-FORMING MATERIAL

(71) Applicants: Yoshiyuki KOYAMA, Tokyo (JP); TOAGOSEI CO., LTD., Tokyo (JP)

(72) Inventors: Ayaka OUCHI, Tokyo (JP); Kenichi NAKAMURA, Tokyo (JP); Yoshiyuki KOYAMA, Tokyo (JP); Tomoko ITO, Funabashi-shi, Chiba (JP)

(73) Assignees: Yoshiyuki KOYAMA, Tokyo (JP); TOAGOSEI CO., LTD., Tokyo (JP)

(21) Appl. No.: 19/140,983

(22) PCT Filed: Dec. 14, 2023
(86) PCT No.: PCT/JP2023/044895

§ 371 Date: Jun. 12, 2026

(30) Foreign Application Priority Data
Dec. 21, 2022 (JP) 2022-204476

(51) Int. Cl. A61L 27/52; A61L 27/16; A61L 27/20; A61L 27/22; A61L 27/26

(52) U.S. Cl. CPC A61L 27/52; A61L 27/16; A61L 27/20; A61L 27/22; A61L 27/26

(57) ABSTRACT

A hydrogel-forming material contains: a polymer (A) which is a tissue-derived biopolymer having a carboxyl group and differing from hyaluronic acid, or a neutralization product thereof, and a polymer (B) having a functional group that can form a hydrogen bond with the carboxyl group (excepting the polymer (A)).

### TECHNICAL FIELD

### Cross-Reference to Related Applications

The present application claims a priority from Japanese Patent Application No. 2022-204476 filed on Dec. 21, 2022, the entirety of the disclosure of which is incorporated herein by reference.

The present disclosure relates to a hydrogel-forming material and, more particularly, to a hydrogel-forming material that can form a hydrogel through contact with water.

### BACKGROUND ART

A hydrogel that can adhere to a biomedical tissue is useful as a variety of medical treatment materials such as an adhesion-preventive material, a hemostatic material, and a wound covering material, and various studies have been conducted in relation to such materials (see, for example, Patent Document 1). Patent Document 1 discloses production of a soft dry gel serving as a hydrogel-forming material by forming a hydrogel via hydrogen bonds between polyacrylic acid and polyvinylpyrrolidone and drying the formed hydrogel. When the hydrogel-forming material disclosed in Patent Document 1 is stuck on a wet biomedical tissue (e.g., a wound or a hemostatic site), the material absorbs a water component such as blood or interstitial fluid to form a gel, thereby achieving adhesion of the material to the biomedical tissue.

### PRIOR ART DOCUMENTS

### Patent Documents

Patent Document 1: Japanese Patent Application Laid-Open (kokai) No. 2014-100462

### SUMMARY OF THE INVENTION

### Problems to be Solved by the Invention

When a hydrogel-forming material is stuck to a biomedical tissue, the hydrogel-forming material placed on the biomedical tissue desirably swells by absorbing water very rapidly (e.g., about several minutes or over ten minutes) so that the water-absorbed hydrogel-forming material (i.e., hydrogel) quickly adheres to the biomedical tissue, whereby a function of protecting a wound, a hemostatic site, or the like and stopping bleeding is rapidly attained. Also, when a hydrogel-forming material is applied to protect an affected part or to other uses, it may be necessary to maintain a gelling state realized by absorption of water on a biomedical tissue continuously (e.g., about 30 minutes to several hours), whereby a state of adhering the hydrogel to the biomedical tissue is continuously maintained for a considerable period of time. Thus, there is demand for a new material meeting such requirements.

The present disclosure has been conceived under the aforementioned circumstances, and an object of the disclosure is to provide a hydrogel-forming material that can rapidly absorb water through contact therewith to swell, and maintain, for a considerably long period of time, a state of swelling resulting from contact with water.

### Means for Solving the Problems

The present inventors have conducted extensive studies to attain the aforementioned object, and have found that the aforementioned problem can be solved by use of a specific polymer serving as one component forming the hydrogel-forming material. Specifically, the present disclosure provides the following means.
[1] A hydrogel-forming material containing a polymer (A) which is a tissue-derived biopolymer having a carboxyl group and differing from hyaluronic acid, or a neutralization product thereof; and a polymer (B) having a functional group that can form a hydrogen bond with the carboxyl group (excepting the polymer (A)).
[2] The hydrogel-forming material as described in [1] above, wherein the tissue-derived biopolymer is at least one species selected from the group consisting of a polysaccharide and a polypeptide.
[3] The hydrogel-forming material as described in [1] or [2] above, wherein the tissue-derived biopolymer is at least one species selected from the group consisting of oxycellulose, carboxyalkylcellulose, pectin, polyglutamic acid, polyaspartic acid, and alginic acid.
[4] The hydrogel-forming material as described in any of [1] to [3] above, wherein the polymer (B) has an amide group. [5] The hydrogel-forming material as described in [4] above, wherein the polymer (B) is at least one species selected from the group consisting of polyvinylpyrrolidone and poly(meth)acrylamide.
[6] The hydrogel-forming material as described in any of [1] to [5] above, which is used as a medical treatment material.

### Advantageous Effects of the Invention

According to the present disclosure, the hydrogel-forming material contains the aforementioned polymer (A) and polymer (B). As a result, the hydrogel-forming material can rapidly absorb water through contact therewith to swell, and maintain, for a considerably long period of time, a state of swelling resulting from contact with water.

### MODES FOR CARRYING OUT THE INVENTION

The present disclosure will next be described in detail. Notably, in the present specification, the term “(meth)acrylate” encompasses acrylate and methacrylate. The term “(meth)acrylic” encompasses acrylic and methacrylic.

### <<Hydrogel-Forming Material>>

The hydrogel-forming material of the present disclosure forms a hydrogel through contact with water. The hydrogel-forming material of the present disclosure contains a polymer (A) which is a tissue-derived biopolymer having a carboxyl group and differing from hyaluronic acid, or a neutralization product thereof; and a polymer (B) having a functional group that can form a hydrogen bond with the carboxyl group (hereinafter may also be referred to as a “functional group E”) (excepting the polymer (A)). Next will be described components contained in the hydrogel-forming material of the present disclosure and optional components which are incorporated in accordance with need.

### <Polymer (A)>

The polymer (A) is a polymer which is produced from a raw material derived from a living body such as a plant or an animal (i.e., a biomass material) or from a biomass-based material, and is a tissue-derived biopolymer having a carboxyl group and/or “—COO—”. However, the polymer (A) differs from hyaluronic acid or a neutralization product thereof. As used herein, the term “biomass material” refers to a bio-resource that can be sustainably reproduced in the presence of sunlight, water, and carbon dioxide (typically, a photosynthetic plant or the like), a bio-resource generated by a microorganism, or a material produced from such a bio-resource. Specific examples of the tissue-derived biopolymer include a “natural-product-derived polymer,” which is a natural product as is or a processed product thereof, a “resource-derived synthetic polymer,” which is produced by polymerizing an organic resource (e.g., L-lactic acid, an amino acid, or the like) as a monomer, and a “microorganism-derived polymer,” which is generated by a microorganism.

The polymer (A) has a carboxyl group and/or “—COO—”. Accordingly, the polymer (A) may be an unneutralized tissue-derived biopolymer still having carboxyl groups; a partial neutralization product of a tissue-derived biopolymer in which a part of the carboxyl groups thereof has or have been neutralized; or a complete neutralization product of a tissue-derived biopolymer in which the entire carboxyl groups thereof have been neutralized. Notably, as used herein, among the polymer (A) species, an unneutralized polymer is referred to as a “carboxyl group-having tissue-derived biopolymer” or a “carboxyl group-containing tissue-derived biopolymer”; and a polymer in which the carboxyl groups have been partially or thoroughly neutralized is referred to as a “neutralization product of a carboxyl group-having tissue-derived biopolymer” or a “neutralization product of a carboxyl group-containing tissue-derived biopolymer.”

When the neutralization product of a carboxyl group-containing tissue-derived biopolymer is employed as a polymer (A), the neutralization product is essentially neutralized at least in a part of the carboxyl group-containing tissue-derived biopolymer. In the neutralization product, examples of the counter ion with respect to “—COO—” include various cations such as lithium ion, sodium ion, potassium ion, magnesium ion, calcium ion, aluminum ion, and ammonium ion. Among them, alkali metal ions are preferred, from the viewpoints of achieving suitable water-swelling performance of the hydrogel-forming material and application to medical uses, with sodium ion or potassium ion being more preferred and sodium ion being still more preferred.

When the polymer (A) is a neutralization product, the degree of neutralization (or neutralization degree) of the polymer (A) is preferably 10 mol % or higher, more preferably 20 mol % or higher, still more preferably 30 mole or higher, yet more preferably 40 mol % or higher, further more preferably 50 mol % or higher, from the viewpoint of securing solubility in an aqueous solvent. Notably, the neutralization degree of the polymer (A) is a value obtained by titrating the polymer solution with aqueous potassium hydroxide to determine the acid number (mgKOH/g) of the polymer, and calculating from the thus-obtained acid number. The specific procedure of determining the acid number is based on the procedure described in the Examples below.

No particular limitation is imposed on the weight average molecular weight of the polymer (A). From the viewpoints of yielding a hydrogel-forming material which can rapidly swell through contact with water, and achieving enhanced performance of handling the polymer (A) or a polymer solution containing the polymer (A), the molecular weight is, for example, 1,800,000 or lower, preferably 1,500,000 or lower. The weight average molecular weight of the polymer (A) may be 1,200,000 or lower, 800,000 or lower, 700,000 or lower, 500,000 or lower, or 300,000 or lower. No particular limitation is imposed also on the lower limit of the weight average molecular weight of the polymer (A), and the lower limit may be 10,000 or higher, or 30,000 or higher.

From the viewpoint of yielding a hydrogel-forming material having excellent water-swelling performance and a hydrogel having excellent adhesion performance to a biomedical tissue, the carboxyl group-containing tissue-derived biopolymer which may be used in the disclosure is preferably at least one species selected from the group consisting of a polysaccharide and a polypeptide. Among them, the carboxyl group-containing tissue-derived biopolymer is preferably at least one species selected from the group consisting of oxycellulose, carboxyalkylcellulose, pectin, polyglutamic acid, polyaspartic acid, and alginic acid. Examples of the carboxyalkylcellulose include carboxymethylcellulose, carboxypropylcellulose.

Oxycellulose is a cellulose derivative formed by oxidizing a cellulose-based raw material with an oxidizing agent. So long as the cellulose-based raw material is a material mainly containing cellulose, examples of the raw material include pulp, natural cellulose, regenerated cellulose, and microcrystalline cellulose (i.e., a mechanically depolymerized cellulose). Also, as the cellulose-based raw material, a commercial product of crystalline cellulose made from pulp as a raw material or the like may be used as is. Examples of the oxidizing agent include 2,2,6,6-tetramethyl-1-piperidine-N-oxyradical (TEMPO) and hypochlorous acid or a salt thereof. Oxidizing of the cellulose-based raw material may be conducted appropriately through a known method.

In the present disclosure, oxycellulose used as a carboxyl group-containing tissue-derived biopolymer may be a fibrous cellulose produced by oxidizing a cellulose-based raw material with an oxidizing agent (i.e., oxycellulose before defibration), or a nano-cellulose produced by oxidizing a cellulose-based raw material with an oxidizing agent and defibrating the thus-formed fibrous cellulose (i.e., oxycellulose after defibration). The concept of the nano-cellulose encompasses cellulose nano-fiber and cellulose nano-crystals.

A commercial product of oxycellulose or a neutralization product thereof may also be used. Examples of the commercial product include AronFibro (registered trademark) (product of Toagosei Co., Ltd.), Cellempia (TEMPO-oxidize CNF, product of Nippon Paper Industries Co., Ltd.), and RHEOCRYSTA (product of DKS Co., Ltd.).

Carboxymethylcellulose (CMC) is a cellulose-based water-soluble polymer in which a carboxymethyl group is incorporated into a part or all of the hydroxy groups of a glucose unit forming the cellulose. Examples of the commercial product of carboxymethylcellulose include Cellogen (product of DKS Co., Ltd.) and CMC Daicel 1110, 1120, 1130, 1140, 1150, and 1160 (products of Daicel Miraizu Ltd.).

Pectin is a polysaccharide present in a plant tissue (e.g., cell wall or middle lamella) and corresponds to polygalacturonic acid including a galacturonic acid unit, and a methyl galacturonate ester unit in which galacturonic acid is partially converted to a methyl ester. Pectin may be recovered from a citrus fruit, apple, beet pulp, etc. through extraction with acid. Examples of the commercial product of pectin include HMpectin and LMpectin (products of Sumitomo Pharma Food and Chemicals, Co., Ltd.), GENUpectin series (products of CP Kelco), and UNIPECTINE series (products of Unitec Foods Co., Ltd.).

Examples of the polyglutamic acid include α-polyglutamic acid and γ-polyglutamic acid. The polyglutamic acid may be composed of one of D-glutamic acid and L-glutamic acid, or both glutamic acids. Polyglutamic acid may be isolated from a product generated obtained from bacterial strains or microorganisms (e.g., Baccilus natto or a microorganism belonging to genus Baccilus). Examples of the commercial product of polyglutamic acid or a neutralization product thereof include Meiji Polyglutamic Acid (product of Meiji Food Materia Co., Ltd.).

Polyaspartic acid may be produced through thermal polymerization of aspartic acid or polymerization of aspartic acid in the presence of a phosphorus catalyst. Polyaspartic acid may be composed of one of D-aspartic acid and L-aspartic acid, or both aspartic acids. Examples of the commercial product of polyaspartic acid or a neutralization product thereof include poly-(α,β)-DL-aspartic acid sodium salt P3418 (product of Sigma-Aldrich).

Alginic acid is a polysaccharide present in marine algae such as brown algae and red algae. Alginic acid may be produced through, for example, acid treatment of marine algae, heating in the presence of an alkali, and extraction. Examples of the algae raw material include kombu and Macrocystis pyrifera. A commercial product of alginic acid or a neutralization product thereof may also be used. Examples of the commercial product of alginic acid include Kimica Algin IL-2, IL-6, I-1, I-3, and I-5 (products of KIMICA Corporation).

### <Polymer (B)>

The polymer (B) has a functional group that can form a hydrogen bond with a carboxyl group (i.e., a functional group E) and differs from the polymer (A). Examples of the functional group E include an amide group, a cyano group, a carbonyl group, an amino group, and a hydroxy group. The functional group E present in the polymer (B) may be the same group or two or more different groups. From the viewpoint of yielding a hydrogel-forming material having excellent water-swelling performance, the functional group E is preferably, among others, an amide group and/or a hydroxy group, particularly preferably an amide group.

Specific examples of the polymer (B), as a polymer having an amide group, include an a polymer including a structural unit derived from an ethylenic unsaturated monomer having an amide group. More specific examples include polymers formed from a monomer such as (meth)acrylamide, N,N-dimethyl(meth)acrylamide, N,N-dimethylaminopropyl (meth)acrylamide, N-methyl(meth)acrylamide, N-vinyl-2-pyrrolidone, or 1-vinyl-4-methyl-2-pyrrolidone, Among them, the polymer having an amide group is preferably at least one species selected from the group consisting of polyvinylpyrrolidone and poly(meth)acrylamide.

Examples of the polymer having a hydroxy group include polyethylene glycol (e.g., Macrogol 4000, Macrogol 6000, or Macrogol 20000, as a commercial product of NOF Corpotaion), polyoxyethylene-hardened castor oil (e.g., Cremophor RH40 (as a commercial product of BASF), HCO-40 and HCO-60 (as commercial products of Nikko Chemicals Co., Ltd.), polyoxyethylenepolyoxypropylene glycol (e.g., Pluronic (registered trademark) F68 (as a commercial product of ADEKA), and poly(vinyl alcohol). Among them, the polymer having a hydroxy group is preferably polyethylene glycol.

Among them, the polymer (B) is preferably at least one species selected from the group consisting of polyethylene glycol, polyoxyethylenepolyoxypropylene glycol, poly(vinyl alcohol), and a polymer having an amide group. Among them, from the viewpoint of yielding a hydrogel-forming material having excellent water-swelling performance upon contact with water, the polymer (B) is preferably a polymer having an amide group, more preferably at least one species selected from the group consisting of polyvinylpyrrolidone and poly(meth)acrylamide. Among them, the polymer (B) is particularly preferably at least one of polyvinylpyrrolidone and polyacrylamide, from the viewpoints of high polymerizability of structure-forming monomers and ease of production of the polymer (B).

Typically, polyvinylpyrrolidone is a polymer formed of N-vinyl-2-pyrrolidone. However, so long as the effects of the present disclosure are not impaired, the polyvinylpyrrolidone may further include a structural unit derived from a monomer other than N-vinyl-2-pyrrolidone (hereinafter may also be referred to as an “additional monomer”). Examples of the additional monomer include an alkyl (meth)acrylate ester, an aliphatic cyclic (meth)acrylate ester, an aromatic (meth)acrylate ester, an alkoxyalkyl (meth)acrylate ester, a hydroxyalkyl (meth)acrylate ester, and a polyalkylene glycol mono(meth)acrylate.

In the polyvinylpyrrolidone, the relative amount of the structural unit derived from an additional monomer or monomers, with respect to all the structural units forming polyvinylpyrrolidone, is preferably 3 mass % or less, more preferably 1 mass % or less, still more preferably 0.5 mass % or less.

Similarly, polyacrylamide is typically a polymer formed of acrylamide. However, so long as the effects of the present disclosure are not impaired, the polyacrylamide may further include a structural unit derived from a monomer other than acrylamide. Specific examples of a monomer other than acrylamide include the compounds and the like exemplified as the aforementioned additional monomer. In the polyacrylamide, the relative amount of the structural unit derived from a monomer other than acrylamide, with respect to all the structural units forming polyacrylamide, is preferably 3 mass % or less, more preferably 1 mass % or less, still more preferably 0.5 mass % or less.

Polymethacrylamide is typically a polymer formed of methacrylamide. However, so long as the effects of the present disclosure are not impaired, the polymethacrylamide may further include a structural unit derived from a monomer other than methacrylamide. Specific examples of the monomer other than methacrylamide include the compounds and the like exemplified as the aforementioned additional monomer. In the polymethacrylamide, the relative amount of the structural unit derived from a monomer other than methacrylamide, with respect to all the structural units forming polymethacrylamide, is preferably 3 mass % or less, more preferably 1 mass % or less, still more preferably 0.5 mass % or less.

From the viewpoint of yielding a hydrogel having more excellent adhesion performance to a biomedical tissue, at least one of a cross-linked polymer or a polymer having a weight average molecular weight of 6,000 or higher (hereinafter may also be referred to as a “high-molecular-weight polymer (BH)”) may be suitably used as the polymer (B), Among them, a high-molecular-weight polymer (BH) may be more suitably used as the polymer (B).

When the high-molecular-weight polymer (BH) is used as the polymer (B), the weight average molecular weight (Mw) of the high-molecular-weight polymer (BH) is preferably 10,000 or higher, more preferably 30,000 or higher, still more preferably 50,000 or higher, from the viewpoints of securing the dynamic strength and thickening effect of the hydrogel and yielding a hydrogel having excellent adhesion performance to a biomedical tissue. Also, from the viewpoint of handling performance of a polymer or its solution, the Mw of the high-molecular-weight polymer (BH) is preferably 100,000, 000 or lower, more preferably 50,000, 000 or lower, still more preferably 30,000, 000 or lower. Notably, the weight average molecular weight of the polymer (B) is a polystyrene-reduced molecular weight value determined through gel permeation chromatography (GPC),

By appropriately combining one or more members of the aforementioned polymer (A) with one or more members of the polymer (B), the hydrogel-forming material of the present disclosure can be produced. From the viewpoints of availability of raw materials, ease of production of the polymer, etc., one specific example of preferred combinations of the polymer (A) and the polymer (B) is the following mode; i.e., the polymer (A) is at least one species selected from the group consisting of oxycellulose, carboxyalkylcellulose, pectin, polyglutamic acid, polyaspartic acid, and alginic acid, or a neutralization product thereof, and the polymer (B) is at least one species selected from the group consisting of polyvinylpyrrolidone and poly(meth)acrylamide.

The total amount of the polymers (A) and (B) contained in the hydrogel-forming material is preferably 70 mass % or more, more preferably 80 mass % or more, still more preferably 90 mass % or more, yet more preferably 95 mass % or more, with respect to the entire amount of the hydrogel-forming material, from the viewpoints of yielding a hydrogel-forming material which has high dynamic strength and can easily swell through contact with water, and yielding a hydrogel having excellent adhesion performance to a biomedical tissue.

The balance in each polymer content of the hydrogel-forming material is preferably adjusted such that the amount of the polymer (B) is 20 to 500 parts by mass with respect to 100 parts by mass of the polymer (A). When the polymer (A) content and the polymer (B) content satisfy the above conditions, there can be formed a hydrogel which exhibits highly improved dynamic strength and excellent adhesion performance to a biomedical tissue, which is preferred. From such viewpoints, the total amount of the polymer (A) and the polymer (B) is more preferably 30 to 400 parts by mass of the polymer (B) with respect to 100 parts by mass of the polymer (A), still more preferably 50 to 300 parts by mass of the polymer (B), yet more preferably 50 to 200 parts by mass of the polymer (B), further more preferably 75 to 150 parts by mass of the polymer (B).

No particular limitation is imposed on the polymerization method for producing the polymer (B). The polymer (B) may be produced by polymerizing a monomer or monomers through a known polymerization method such as solution polymerization, suspension polymerization, emulsion polymerization, or bulk polymerization. In one procedure of solution polymerization, an organic solvent and a monomer or monomers are fed to a reactor, and a polymerization initiator (e.g., an azo compound) is added to the mixture, followed by heating the reaction system at 40 to 250° C. for polymerization, to thereby yield a target polymer.

### <Additional Components>

The hydrogel-forming material may further contain an additional component in accordance with the purpose of use and the like. Examples of the additional component include hyaluronic acid or a neutralization product thereof. When the hydrogel-forming material is applied to medical uses and the like, various pharmaceutical agents such as an antibacterial agent, an anti-inflammatory agent, a blood coagulant, an anti-coagulant, a local anesthetic, a vasoconstrictor, and a vasodilator may be incorporated into the hydrogel-forming material as additional components. These additional components may be incorporated singly or in combination of more species. The amount of the additional component may be appropriately tuned, so long as the effects of the present disclosure are not impaired.

When the hydrogel-forming material contains hyaluronic acid or a neutralization product thereof, the relative amount of hyaluronic acid or a neutralization product thereof, with respect to 100 parts by mass of the total amount of the polymer (A) and the polymer (B), is preferably adjusted to 0.01 to 20 parts by mass. By tuning the relative amount of hyaluronic acid or a neutralization product thereof to fall within the above range, water retention of the hydrogel can be improved, while suitable softness of the hydrogel-forming material is maintained. From such viewpoints, the relative amount of hyaluronic acid or a neutralization product thereof, with respect to 100 parts by mass of the total amount of the polymer (A) and the polymer (B), is more preferably 0.1 parts by mass or more, still more preferably 0.5 parts by mass or more. The upper limit of the relative amount of hyaluronic acid or a neutralization product thereof, with respect to 100 parts by mass of the total amount of the polymer (A) and the polymer (B), is more preferably 15 parts by mass or less, still more preferably 10 parts by mass or less. Hyaluronic acid or a neutralization product thereof may be used singly or in combination of two or more species.

### <Method of Producing Hydrogel-Forming Material>

No particular limitation is imposed on the method for producing the hydrogel-forming material of the present disclosure. The hydrogel-forming material of the present disclosure may be yielded through, for example, any of the following methods [1] to [3].
Method [1]: Bringing a film-shape solid containing one of the polymer (A) and the polymer (B) in contact with a solution containing the other polymer, and then drying.
Method [2]: Mixing a solution containing the polymer (A) with a solution containing the polymer (B) in the presence of hyaluronic acid or a neutralization product thereof, and removing the solvent.
Method [3]: Mixing an alcoholic solution containing the polymer (A) with an alcoholic solution containing the polymer (B), and removing the solvent.

When the manner of simply mixing an aqueous solution of the polymer (A) with an aqueous solution of the polymer (B) is employed, hydrogen bonds between functional groups of the polymer (A) and those of the polymer (B) are very rapidly formed. However, in some cases, fibrous aggregates may be formed to thereby impede formation of a hydrogel. In addition, even if a hydrogel is yielded, the resultant hydrogel has insufficient water-solubility and water-swelling performance, and adhesion performance to a biomedical tissue is poor. In contrast, through employment of any of the aforementioned methods [1], [2], and [3], a hydrogel-forming material exhibiting excellent water-solubility and water-swelling performance can be successfully produced.

### Method [1]

In the method [1], firstly, a film-shape solid containing one of the polymer (A) and the polymer (B) (hereinafter may also be referred to as a “first polymer”) is produced. Examples of the method of producing the film-shape solid include solution drying and hot pressing. Of these, solution drying is preferred, since bubble formation can be suppressed, to thereby form a flat film. In a preferred mode of producing a film-shape solid through solution dying, the first polymer is dissolved in a solvent to prepare a polymer solution (hereinafter may also be referred to as a “first polymer solution”), and then the first polymer solution is applied to a support, followed by drying. Notably, the first polymer forming the film-shape solid may be the polymer (A) or the polymer (B).

Examples of the solvent for dissolving the first polymer include water, a mixture of water and an organic solvent which can be dissolved in water, and an organic solvent which can be dissolved in water. Examples of the organic solvent which can be dissolved in water include methanol, ethanol, and acetone. Among them, water, ethanol, or a water-ethanol liquid mixture is preferred as the solvent for dissolving the first polymer. No particular limitation is imposed on the polymer concentration of the first polymer solution, and the concentration is, for example, 0.01 to 10 mass %, preferably 0.1 to 5 mass %.

No particular limitation is imposed on the method of forming the film-shape solid on a substrate, and a known film-formation method may be appropriately employed. In one example, a first polymer solution is applied onto a substrate and, preferably, heating is conducted to remove the solvent, to thereby form a film-shape solid containing the first polymer on the substrate. When the heating treatment is conducted, the heating temperature is, for example, 50 to 120° C., and the heating time is, for example, 0.1 to 30 hours. Alternatively, the heating treatment may be conducted under reduced pressure or air blowing. The thickness of the film-shape solid formed on a substrate is, for example, 1 to 5,000 μm. The water content of the film-shape solid is, for example, 10 mass % or less.

Subsequently, the film-shape solid which has been formed on the substrate is brought into contact with a solution prepared by dissolving the other polymer (i.e., the polymer (A) or the polymer (B) differing from the first polymer) (hereinafter may also be referred to as a “second polymer”) in a solvent (hereinafter, the solution may also be referred to as a “second polymer solution”). Examples of the solvent for dissolving the second polymer include the same as exemplified in relation to the solvent for dissolving the first polymer. The polymer concentration of the second polymer solution is, for example, 0.1 to 30 mass %, preferably 1 to 20 mass %.

No particular limitation is imposed on the method of bringing the film-shape solid containing the first polymer into contact with the second polymer solution. Examples of the method of bringing the film-shape solid into contact with the second polymer solution include a method in which a second polymer solution is applied onto, added dropwise to, or sprayed to the surface of a film-shape solid; and a method in which a film-shape solid is immersed in a second polymer solution. In one preferred mode, a second polymer solution is formed on the surface of a film-shape solid through, for example dropwise addition, to thereby form a liquid layer formed of the second polymer solution on the film-shape solid, and the coated substrate is allowed to stand for a specific period of time (e.g., 10 to 180 minutes). No particular limitation is imposed on the thickness of the liquid layer and is, for example, 0.1 to 50,000 μm. Thus, the first polymer present in the film-shape solid is gradually dissolved in the second polymer solution, to thereby form a hydrogel.

In the case of contact of the film-shape solid containing the first polymer with the second polymer solution, the amount of the second polymer solution to be in contact with the film-shape solid is preferably predetermined such that a cross-linked structure is formed to an appropriate extent in the formed hydrogel. Specifically, the amounts of the film-shape solid and the second polymer solution and the polymer concentration are preferably adjusted such that the amount by mole of the functional group E present in the polymer (B), with respect to 1 mol (in total) of the carboxyl group and “—COO—” present in the polymer (A) is preferably tuned to 0.1 to 10 mol, more preferably 0.2 to 8 mol, still more preferably 0.5 to 2 mol.

In the case of providing, as a hydrogel-forming material, a dry product of hyaluronic acid or a neutralization product thereof, hyaluronic acid or a neutralization product thereof may be contained in the film-shape solid, or in the second polymer solution. Of these, from the viewpoint of achieving suitable formation of a hydrogel, the mode in which the second polymer solution contains hyaluronic acid or a neutralization product thereof is preferred. When the hyaluronic acid or a neutralization product thereof is contained in the second polymer solution, in an alternative manner, hyaluronic acid or a neutralization product thereof is incorporated into the second polymer solution in advance, and the second polymer solution containing hyaluronic acid or a neutralization product thereof is brought into contact with the film-shape solid. In a yet alternative manner, the film-shape solid is brought into contact with the second polymer solution, and then hyaluronic acid or a neutralization product thereof is added to the second polymer solution. Of these, from the viewpoint of achieving suitable formation of a hydrogel, the mode in which the second polymer solution in advance containing hyaluronic acid or a neutralization product thereof is brought into contact with the film-shape solid is preferred.

In the case of contact of the second polymer solution containing hyaluronic acid or a neutralization product thereof with the film-shape solid, the relative amount of the hyaluronic acid or a neutralization product thereof in the second polymer solution, with respect to 100 parts by mass of the second polymer, is preferably adjusted 0.01 to 30 parts by mass, more preferably 0.1 to 20 parts by mass, still more preferably 0.5 to 15 parts by mass.

Thereafter, the thus-formed hydrogel; i.e., a product obtained by bringing the film-shape solid containing the first polymer into contact with the second polymer solution (hereinafter may also be referred to as a “hydrogel product”) is dried, whereby a dry product of interest is yielded as a hydrogel-forming material. No particular limitation is imposed on the method of drying a hydrogel, and a known drying treatment technique may be appropriately employed.

For example, in the case of drying a hydrogel product through solution drying, drying is preferably conducted through lyophilization. In lyophilization, the freezing temperature is, for example, −70° C. to −5° C., preferably −60° C. to −5° C. Drying via lyophilization is preferably conducted at room temperature under reduced pressure. The pressure during lyophilization is, for example, 50 Pa or lower, preferably 20 Pa or lower, more preferably 10 Pa or lower.

In the case of producing a sponge-like hydrogel-forming material, preferably, lyophilization involves transferring a hydrogel product to be in a supercooling state, and then frozen. In this case, the hydrogel product is preferably cooled through a plurality of steps having different cooling temperatures. According to the above procedure, a sponge-like hydrogel-forming material which has high dynamic strength and excellent adhesion performance to a biomedical tissue in a water-swelling state can be produced in a relatively easy manner.

Specifically, in a preferred manner, a hydrogel product is cooled at −10° C. to 0° C., to thereby attain a supercooling state of the hydrogel product, in a first cooling treatment, and then, in a second cooling treatment, the hydrogel product is cooled at a temperature lower than −10° C., to thereby freeze the hydrogel product. In the first cooling treatment, the temperature is preferably lowered gradually, from the viewpoint of efficiently attaining a supercooling state. In a second cooling treatment, from the viewpoint of yielding a hydrogel-forming material which has high dynamic strength and excellent adhesion performance to a biomedical tissue in a water-swelling state, the cooling temperature is preferably adjusted to be −15° C. or lower, more preferably −20° C. or lower, still more preferably −25° C. or lower. No particular limitation is imposed on the cooling time in each of the first and second cooling treatment steps, and the cooling temperature may be appropriately set, for example, to 3 minutes to 5 hours. In the first cooling treatment step and/or the second cooling treatment step, cooling treatment may be conducted through multiple steps having different cooling temperatures.

Notably, as used herein, the term “dry” encompasses a state in which water has been completely removed, and a state in which water remains in the course of drying. The water content of the dry product after the drying treatment is, for example, 10 mass % or lower, preferably 5 mass % or lower.

Through the aforementioned procedure, a hydrogel product obtained by bringing the film-shape solid containing one of the polymer (A) and the polymer (B) into contact with a solution containing the other polymer is dried, to thereby form a dry product of the hydrogel.

### Method [2]

In the method [2], a solution containing the polymer (A) is mixed with a solution containing the polymer (B) in the presence of hyaluronic acid or a neutralization product thereof, and removing the solvent from the resultant solution, to thereby yield a dry product serving as a hydrogel-forming material.

In the solution containing the polymer (A) (hereinafter may also be referred to as a “polymer solution A”), and the solution containing the polymer (B) (hereinafter may also be referred to as a “polymer solution B”), examples of the solvent that can dissolve the polymer include the same solvents as exemplified in relation to the solvent that can dissolve the first polymer. Among them, from the viewpoint of conducting the drying step at high efficiency, water is preferably used as a sole solvent. In the polymer solutions A and B, the polymer concentration is, for example, 0.001 to 5 mass %, preferably 0.01 to 1 mass %.

In the polymer solutions A and B, each of the polymer (A) content and the polymer (B) content is preferably adjusted such that the polymer (B) content is 20 to 500 parts by mass with respect to 100 parts by mass of the polymer (A), by regulating the amounts and concentrations of the polymer solutions A and B. The amount of each of the polymer (A) and the polymer (B), with respect to 100 parts by mass of the polymer (A), is more preferably adjusted so as to be the polymer (B) content of 30 to 400 parts by mass, still more preferably 50 to 300 parts by mass.

In the method [2], the amount of use of hyaluronic acid or a neutralization product thereof, with respect to 100 parts by mass of the polymer (A), is preferably adjusted to 0.01 to 30 parts by mass, more preferably 0.1 to 30 parts by mass, still more preferably 0.5 to 20 parts by mass. Hyaluronic acid or a neutralization product thereof is preferably used in the form of aqueous solution.

Subsequently, the thus-obtained liquid mixture containing the polymer (A), the polymer (B), and hyaluronic acid or a neutralization product thereof is subjected to a drying treatment for the removal of solvent, to thereby yield a target dry product. The drying treatment is preferably conducted as a drying by lyophilization. Lyophilization may be conducted through a customary method. In one exemplary procedure, the aforementioned liquid mixture is poured into a mold and frozen, and the thus-molded frozen product is lyophilized, to thereby yield a target product (dry product) having a shape of interest. Preferably, the lyophilization treatment is conducted after the hydrogel product has been transferred to be in a supercooling state, followed by freezing. Details of the procedure are the same as have been described in relation to the method [1].

### Method [3]

In the method [3], an alcoholic solution containing the polymer (A) is mixed with an alcoholic solution containing the polymer (B), and then the solvent is removed, to thereby produce a dry product serving as a hydrogel-forming material. Notably, in the method [3], a satisfactory-quality hydrogel-forming material can be yielded, unless mixing of the polymer solutions is conducted in the presence of hyaluronic acid or a neutralization product thereof. Thus, the method [3] is advantageous.

In the alcoholic solution containing the polymer (A) (hereinafter may also be referred to as an “alcoholic solution A”) and the alcoholic solution containing the polymer (B) (hereinafter may also be referred to as an “alcoholic solution B”), a C1 to C5, linear-chain or branched alcohol is preferably used as the aforementioned alcohol. Notably, the alcohol used in preparation of the alcoholic solutions A and B may be the same species or different species. In preparation of each of the alcoholic solutions A and B, one single alcohol or two or more alcohols may be used.

From the viewpoint of yielding a hydrogel exhibiting excellent adhesion performance to a biomedical tissue, the alcohol used in preparation of the alcoholic solutions A and B is preferably, among others, a C1 to C3 linear-chain or branched alcohol. Also, in consideration of planning it to medical uses, among others, at least one species selected from the group consisting of ethanol, n-propanol, and isopropanol is preferred, with ethanol being particularly preferred. The polymer concentration of each of the alcoholic solutions A and B is, for example, 0.1 to 30 mass %, preferably 0.5 to 20 mass %.

In the alcoholic solutions A and B, each of the polymer (A) content and the polymer (B) content is preferably adjusted by controlling the amount and concentration of the alcoholic solutions A and B such that the relative amount of the polymer (B) is adjusted to 20 to 500 parts by mass with respect to 100 parts by mass of the polymer (A). The relative amount of the polymer (A) or the polymer (B) is more preferably adjusted such that the relative amount of the polymer (B) is adjusted to 30 to 400 parts by mass, still more preferably 50 to 300 parts by mass, with respect to 100 parts by mass of the polymer (A). Also, in the case of producing a hydrogel-forming material containing hyaluronic acid or a neutralization product thereof, hyaluronic acid or a neutralization product thereof may be incorporated into at least one of the alcoholic solution A and the alcoholic solution B. The relative amount of hyaluronic acid or a neutralization product thereof in the alcoholic solution A or the alcoholic solution A or both is, for example, 0.01 to 15 parts by mass, with respect to 100 parts by mass of the polymer.

Subsequently, the above-prepared liquid mixture containing the polymer (A), the polymer (B), and alcohol is subjected to a drying treatment for removing the solvent, to thereby yield a target dry product. No particular limitation is imposed on the drying method, and, for example, natural drying may be employed, or one or more means of heating, blowing, etc. in combination. Alternatively, lyophilization may also be employed. Details of the lyophilization treatment are the same as described in relation to the methods [1] and [2].

### <Physical Properties of Hydrogel-Forming Material>

The thus-formed hydrogel-forming material is not dissolved or hardly dissolved in an ionic buffer such as body fluid and appropriately swells through contact with water. When the hydrogel-forming material is dried by means of a non-blow drier at 150° C. for 60 minutes, a “degree of water-swelling θ,” which is a ratio of W₂ to W₁ is determined, wherein W₁ represents the mass (g) of hydrogel-forming material after drying, and W₂ represents the mass (g) of the hydrogel-forming material after swelling with an ionic buffer for a predetermined period of time. In this case, the degree of water-swelling θ is represented by the following numerical expression (1):

$$Water - swelling degree θ = {W}_{2} / {W}_{1} \qquad ( 1 )$$

wherein W₁ represents a mass (g) after drying by means of a non-blow drier at 150° C. for 60 minutes, and
W₂ represents a mass (g) after swelling with ionic buffer for a predetermined period of time.

From the viewpoint of yielding a hydrogel-forming material that can rapidly absorb water through contact with water and rapidly develop adhesion performance to a biomedical tissue, the hydrogel-forming material of the present disclosure preferably exhibits a degree of water-swelling θ after swelling with ionic buffer for 10 minutes (hereinafter may also denoted by “water-swelling degree θ₁₀”) of 3 or higher, more preferably 5 or higher, still more preferably 8 or higher. Also, the upper limit of the water-swelling degree fic after swelling with ionic buffer for 10 minutes is preferably 60 or lower, from the viewpoint of suppressing an excessive increase in the volume of the water-absorbed hydrogel-forming material (i.e., a hydrogel), to thereby suppress oppression to a biomedical tissue, more preferably 55 or lower, still more preferably 50 or lower.

After the hydrogel-forming material of the present disclosure is in a water-swelling state after absorption of water through contact with water, the water-swelling state can be maintained for a considerably long period of time. As a result, after completion of adhesion to a biomedical tissue via water absorption, the hydrogel-forming material of the present disclosure can maintain a state of contact with a biomedical tissue for a considerably long period of time (e.g., about 30 minutes to several hours), to thereby protect the biomedical tissue. Specifically, the hydrogel-forming material of the present disclosure preferably exhibits a degree of water-swelling θ after swelling with ionic buffer for 60 minutes (hereinafter may also denoted by “water-swelling degree θ₆₀”) of 2 or higher, more preferably 4 or higher, still more preferably 5 or higher. Also, the upper limit of the water-swelling degree 06 after swelling with ionic buffer for 60 minutes is preferably 50 or lower, more preferably 40 or less, still more preferably 30 or less, from the viewpoint of suppressing an excessive increase in the volume of the water-absorbed hydrogel-forming material, to thereby suppress oppression to a biomedical tissue.

### <Mode of Use of Hydrogel-Forming Material>

Before use, the hydrogel-forming material of the present disclosure is a dry solid (i.e., a dry product) and is converted to a hydrogel (i.e., a swollen body) through swelling by absorbing water in contact with water. Examples of the water include water itself, an organic solvent which can be dissolved water (e.g., ethanol), a body fluid (e.g., blood or interstitial fluid), and a liquid mixture thereof.

No particular limitation is imposed on the shape of the hydrogel-forming material of the present disclosure (dry product), and examples of the shape include film, sheet, sponge, and powder. Also, no particular limitation is imposed on the dimensions of the dry product. When the dry product obtained from the hydrogel-forming material is a film, the thickness of the dry product is generally about 0.1 to about 50,000 μm.

The hydrogel-forming material of the present disclosure may be provided as a supported-on-substrate product, a package product with film or the like, or a spray product. When the hydrogel-forming material of the present disclosure is provided as a supported-on-substrate product, no particular limitation is imposed on the shape and the material of the support, and examples of the material include fabrics such as woven fabric and nonwoven fabric; and resin substrates such as those made of polystyrene, polypropylene, or polyethylene. The hydrogel-forming material of the present disclosure, having high dynamic strength and excellent softness, is preferably used as a hydrogel-forming film or a hydrogel-forming sponge.

Before contact with water, the hydrogel-forming material of the present disclosure assumes a dry product having softness. Through contact with water, the dry product converts to a swelling form, by which adhesion to a biomedical tissue is achieved. Also, the hydrogel-forming material of the present disclosure is not absorbed by a living body and is gradually degraded to be solubilized under physiological conditions. As a result, the hydrogel-forming material of the present disclosure has high safety and can be left in the living body. The hydrogel-forming material of the present disclosure having such characteristics is suitable for a medical treatment material. Specifically, the material is particularly suited for medical treatment materials such as an adhesion-preventive material, a hemostatic material, and a wound covering material.

### EXAMPLES

The present disclosure will next be described in detail by way of example, which should not be construed as limiting the disclosure thereto. Hereinafter, unless otherwise specified, the units “part(s)” and “%” encompass “part(s) by mass” and “mass %,” respectively.

The neutralization degree of a polymer used in production of the hydrogel-forming material was determined through the following procedure.

### (Neutralization Degree of Polymer)

A polymer of interest (0.2 g) was placed in a polypropylene cup and precisely weighed. Tetrahydrofuran (THF) (about 50 mL) was added thereto, and the mixture was stirred by means of a magnetic stirrer, to thereby prepare a solution. Pure water (about 5 mL) was added to the thus-obtained polymer solution, to thereby prepare a measurement sample. The sample was titrated with 0.1-mol/L potassium hydroxide/ethanol solution by means of an automated titration apparatus (COM-A19, product of HIRANUMA), whereby the acid number (mgKOH/g) of the polymer was determined. From the determined acid number, the neutralization degree (mol %) of the polymer was calculated.

### <Production of Hydrogel-Forming Material>

### Example 1

To polyvinylpyrrolidone (polymer (B), hereinafter may also be abbreviated as “PVP”) (2.0 g) and sodium hyaluronate (hereinafter may also be abbreviated as “HA”) (0.26 g), water was added, and the total volume of the mixture was adjusted to 50 mL, to thereby prepare an aqueous solution (45 mL). Then, the aqueous solution (45 mL) and a 4.63-mass % aqueous solution (51 mL) of 90 mol %-neutralized sodium polyglutamate (polymer (A), hereinafter may also be denoted by “90 mol %-neutralized PGluNa”) were mixed, to thereby prepare a solution mixture.

Separately, a silicone rubber sheet having an opening (25 mm×7 mm) (thickness: 10 mm) was placed on a polypropylene support (50 mm×50 mm), and the above-prepared solution mixture (1.5 mL) containing PVP, HA, and 90 mol %-neutralized PGluNa was cast onto the support. Preliminary cooling (0° C.×0.5 h, then −4° C.×0.5 h) and cooling (−35° C.×1 h) were sequentially conducted, to thereby freeze the product.

The frozen product was subjected to lyophilization at room temperature under reduced pressure (5 Pa), to thereby yield a hydrogel-forming material (dimensions: 25 mm×7 mm×7 mm). The proportions of the mixed polymers; i.e., 90 mol %-neutralized PGluNa: PVP: HA, were 1:0.75:0.1 (by mass).

### Example 2

The procedure of Example 1 was repeated, except that the polymer (A) aqueous solution was altered to a 3.42 mass % aqueous polymer solution containing 30 mol %-esterified pectin, and the proportions of the mixed polymers; i.e., 30 mol %-esterified pectin: PVP: HA, were modified to 1:0.54:0.07 (by mass), to thereby yield a hydrogel-forming material.

### Example 3

A silicone rubber sheet having an opening (25 mm×7 mm) (thickness: 10 mm) was placed on a polypropylene support (50 mm×50 mm), and a 5.13 mass % aqueous solution (1.5 mL) of 100 mol %-Na-neutralized oxycellulose (polymer (A)) was cast onto the support, followed by drying at 70° C. for 20 hours, to thereby prepare a 100 mol %-Na-neutralized oxycellulose film. Subsequently, a mixture (1.5 mL) of a 4.6-mass % aqueous solution (0.6 mL) of PVP (polymer (B)) and a 0.4-mass % aqueous solution (0.9 mb) of HA was added dropwise to the surface of the 100 mol %-Na-neutralized oxycellulose film, and the film was allowed to stand for 60 minutes. Preliminary cooling (0° C.×0.5 h, then −4° C.×0.5 h) and cooling (−35° C.×1 h) were sequentially conducted, to thereby freeze the product.

The frozen product was subjected to lyophilization at room temperature under reduced pressure (5 Pa), to thereby yield a hydrogel-forming material (dimensions: 25 mm×7 mm×7 mm). The proportions of the mixed polymers; i.e., 100 mol %-Na-neutralized oxycellulose: PVP: HA, were 1:0.36:0.05 (by mass).

### Example 4

The procedure of Example 3 was repeated, except that raw materials were changed as shown in Table 1, to thereby yield a hydrogel-forming material.

### Example 5

The procedure of Example 3 was repeated, except that the polymer (A) aqueous solution was altered to a 3.42 mass % aqueous polymer solution containing 30 mol %-esterified pectin, and the proportions of the mixed polymers; i.e., 30 mol %-esterified pectin: PVP: HA, were modified to 1:0.54:0.07 (by mass), to thereby yield a hydrogel-forming material.

### Comparative Example 1

A silicone rubber sheet having an opening (25 mm×7 mm) (thickness: 10 mm) was placed on a polypropylene support (50 mm×50 mm), and a 1.2 mass % aqueous solution (1.5 mL) of uncross-linked poly(acrylic acid) (hereinafter may also be abbreviated as “PAA”) was cast onto the support, followed by drying at 70° C. for 20 hours, to thereby prepare a PAA film. Subsequently, a mixture (1.5 mL) of a 4.6-mass % aqueous solution (0.6 mL) of PVP (polymer (B)) and a 0.4-mass % aqueous solution (0.9 mb) of HA was added dropwise to the surface of the PAA film, and the film was allowed to stand for 60 minutes. Preliminary cooling (0° C.×0.5 h, then-4° C.×0.5 h) and cooling (−35° C.×1 h) were sequentially conducted, to thereby freeze the product.

The frozen product was subjected to lyophilization at room temperature under reduced pressure (5 Pa), to thereby yield a hydrogel-forming material (dimensions: 25 mm×7 mm×7 mm). The proportions of the mixed polymers; i.e., PAA: PVP: HA, were 1:1.53:0.2 (by mass).

### Evaluation

The degree of swelling with water θ (hereinafter may also be referred to as a water-swelling degree) of each of the hydrogel-forming materials prepared in Examples 1 to 5 and Comparative Example 1 was determined, by the following equation (1):

$$Water - swelling degree θ = {W}_{2} / {W}_{1} \qquad ( 1 )$$

wherein W₁ represents a mass (g) of a hydrogel-forming material after drying by a non-blow drier at 150° C. for 60 minutes, and W₂ represents a mass (g) of a hydrogel-forming material after swelling with ionic buffer for a specific time.

More specifically, water-swelling degree θ was determined through the following procedure. Table 1 shows the results.

### Determination of Water-Swelling Degree θ

Each of the above-prepared hydrogel-forming materials was dried by means of a non-blow drier (DX-40, product of Yamato Scientific Co., Ltd.) at 150° C. for 60 minutes, and the mass of the hydrogel-forming material (W₁ in the above equation (1), unit: g) was measured. Next, the hydrogel-forming material after drying was placed into an ionic buffer; a phosphate buffer (0.1-mol/L phosphate buffer, pH 7.2, product of FUJIFILM Wako Pure Chemical Corp.), and was allowed to stand for 10 minutes. After immersion in the ionic buffer for 10 minutes, the mass of the hydrogel-forming material (W₂ in the above equation (1), unit: g) was measured. Water-swelling degree θ₁₀ of the hydrogel-forming material after swelling with ionic buffer for 10 minutes was calculated by the aforementioned equation (1).

Also, the same procedure as described above was repeated, except that the time of allowing the hydrogel-forming material to stand in the phosphate buffer after placing was changed from 10 minutes to 60 minutes, and the mass of the hydrogel-forming material (W₂ in the above equation (1), unit: g) was measured after immersion in the ionic buffer for 60 minutes, to thereby calculate the water-swelling degree θ₆₀ of the hydrogel-forming material after swelling with ionic buffer for 60 minutes.

TABLE 1

| | | | | Evaluation | |
|---|---|---|---|---|---|
| | Hydrogel-forming material | | | Water-swelling degree θ (g/g) | |
| | | Polymer | Additional | 10 minutes | 60 minutes |
| | Polymer (A) | (B) | component | (θ₁₀) | (θ₆₀) |
| Example 1 | 90 mol %-neutralized | PVP | HA | 9.3 | 8.4 |
| | PGluNa | | | | |
| Example 2 | 30 mol %-esterified pectin | PVP | HA | 12.7 | 8.1 |
| Example 3 | 100 mol %-Na-neutralized | PVP | HA | 12.0 | 7.1 |
| | oxycellulose | | | | |
| Example 4 | Unneutralized oxycellulose | PVP | HA | 7.6 | 6.9 |
| Example 5 | 30 mol %-esterified pectin | PVP | HA | 10.5 | 7.3 |
| Comparative | PAA | PVP | HA | 10.1 | 1.7 |
| Example 1 | | | | | |

Details of the compounds shown in Table 1 are as follows.
90 mol %-neutralized PGluNa: sodium polyglutamate (neutralization degree: 90 mol %, weight average molecular weight: 1,000, 000, Meiji Polyglutamic Acid, product of Meiji Food Materia Co., Ltd.)
30 mol %-esterified pectin: methyl esterified pectin (H&F pectin Classic AF 701, esterification degree: 30 mol %, weight average molecular weight: 200,000, product of Sumitomo Pharma Food and Chemicals, Co., Ltd.)
100 mol %-Na-neutralized oxycellulose: 100 mol %-neutralized sodium salt of oxycellulose (AronFibro, neutralization degree: 100 mol %, weight average molecular weight: 100,000, product of Toagosei Co., Ltd.)
Unneutralized oxycellulose: unneutralized product of oxycellulose (AronFibro, product of Toagosei Co., Ltd.).
PAA: Uncorss-linked poly(acrylic acid) (Jurymer AC-10LHPK, weight average molecular weight: 1,200,000, product of Toagosei Co., Ltd.)
PVP: polyvinylpyrrolidone (Kollidon 90F, polystyrene-reduced weight average molecular weight: 320,000 (dimethylformamide elutate), product of BASF).
HA: sodium hyaluronate (Hyaluronic acid HA-LQH, weight average molecular weight: 1,200,000, product of Kewpie Corporation)

### Results of Evaluation

As is clear from Table 1, hydrogel-forming materials (Examples 1 to 5), each containing the polymer (A) (i.e., a tissue-derived biopolymer having a carboxyl group or a neutralization product thereof) and the polymer (B) (i.e., a polymer having functional group that can form a hydrogen bond with a carboxyl group), were found to exhibit a high water-swelling degree θ (θ₁₀ and θ₆₀) after swelling with water for 10 minutes and 60 minutes, respectively). Thus, consequently, the hydrogel-forming materials of Examples 1 to 5 can rapidly absorb water such as blood or interstitial fluid to swell, and maintain the swelling state for a relatively long period of time.

In contrast, the hydrogel-forming material of Comparative Example 1, employing an uncross-linked poly(acrylic acid) as a carboxyl group-having polymer, instead of the polymers (A) of Examples 1 to 5, was found to exhibit a lower water-swelling degree 060 after swelling with water for 60 minutes, as compared with Examples 1 to 5, indicating rapid disappearance of a water-swelling state, although the water-swelling degree die after swelling with water for 10 minutes was equivalent to that obtained in Examples 1 to 5.

As a result, according to a hydrogel-forming material, containing the polymer (A) (i.e., a tissue-derived biopolymer having a carboxyl group or a neutralization product thereof) and the polymer (B) (i.e., a polymer having functional group that can form a hydrogen bond with a carboxyl group), rapid water-absorbing performance upon contact with water can be achieved, and the water-swelling state realized after contact with water can be maintained for a relatively long period of time.

The present invention should not be limited to the aforementioned embodiments and encompasses various variations and modifications falling within equivalents, so long as they do not deviate from the gist of the present invention. Thus, those skilled in the art should understand that, with reference to the aforementioned indications, various combinations and modes, and further, one element or other combinations of more or less thereof also fall within the scope and concept of the present invention.

1. A hydrogel-forming material comprising a polymer (A) which is a tissue-derived biopolymer comprising a carboxyl group and differing from hyaluronic acid, or a neutralization product thereof; and
a polymer (B) comprising a functional group that can form a hydrogen bond with the carboxyl group, excepting the polymer (A).

2. The hydrogel-forming material according to claim 1, wherein the tissue-derived biopolymer is at least one species selected from the group consisting of a polysaccharide and a polypeptide.

3. The hydrogel-forming material according to claim 1 or 2, wherein the tissue-derived biopolymer is at least one species selected from the group consisting of oxycellulose, carboxyalkylcellulose, pectin, polyglutamic acid, polyaspartic acid, and alginic acid.

4. The hydrogel-forming material according to claim 1 or 2, wherein the polymer (B) comprises an amide group.

5. The hydrogel-forming material according to claim 4, wherein the polymer (B) is at least one species selected from the group consisting of polyvinylpyrrolidone and poly(meth)acrylamide.

6. The hydrogel-forming material as recited in claim 1 or 2, which is used as a medical treatment material.
